(19) United States
(12) Patent Application Publication
(10) Pub. No.: US 2007/0055257 A1
(43) Pub. Date: Mar. 8, 2007

(54) Cannulated screw access system

(76) Inventors: Alex Vaccaro, Gladwyne, PA (US); Richard W. Woods, Catonsville, MD (US); Kevin R. Strauss, Columbia, MD (US); Todd M. Wallenstein, Ashburn, VA (US)

Correspondence Address: MANELLI DENISON & SELTER, 2000 M STREET NW SUITE 700, WASHINGTON, DC, 20036-3307, US

(21) Appl. No.: 11/476,775

(22) Filed: Jun. 29, 2006

Related U.S. Application Data
(60) Provisional application No. 60/695,083, filed on Jun. 30, 2005.

(51) Int. Cl. A61B 17/58

(52) U.S. Cl. 606/73

(57) ABSTRACT

The present invention provides a novel minimally invasive surgical system and method for introducing instruments and/or biomaterial into the interior of a bone, particularly the interior of a vertebral body, using a unique cannulated screw that is sized and configured to penetrate the cortical bone and thereby provide access to the interior of the bone through the integral cannula of the screw. That same screw having a screw head that is then adaptable to employ a connector for securely holding a connecting device, such as a spinal rod or plate that can then be used to fix bones or bone fragments together. A method of fixing bones or bone fragments is also provided.

### BACKGROUND OF THE INVENTION

1. Field of the Invention

The present invention relates to orthopedic surgery, and in particular to devices, systems and methods for minimally invasive access into the bone and particularly minimally invasive access to the vertebral body for biomaterial delivery and fixation of the spine. More particularly, the present invention provides a cannulated screw access system to facilitate the delivery of materials into the vertebral body, the screw head being attachable or adaptable to connection to a spinal rod or other appliance for the fixation of the spine.

2. Background of the Technology

Spinal fixation implants and instrument are widely employed in surgical procedures for correcting spinal injuries and diseases. The need to improve patient outcome and decrease patient length of stay has led to minimally invasive surgical procedures. Minimally invasive procedures are typically governed by a very small skin incision allowing for far less exposure of the patient thereby decreasing the risk of infection and decreasing the amount of general trauma due to the procedure.

Before spinal fixation can occur, the injured or diseased anatomy must be addressed. Typically, a single level spin fusion requires the partial or complete removal of the disc between two vertebrae in a procedure called a discectomy. The void created by the discectomy is then filled with an implant, bone graft, artificial bone graft, artificial bone graft or some combination therein. A series of bone screws and rods or other apparatus are used to stabilize the area until the fusion occurs.

When disease or injury, such as osteoporosis, results in damage to a vertebral body a means to repair the bone structures is required. Various procedures and apparatus have been developed to address the damaged vertebral body and are described below.

Vertebroplasty is a procedure whereby the vertebra is structurally reinforced using “bone cement,” a compound of polymethylmethacrylate (PMMA). PMMA is injected into the vertebral body which has become more porous typically due to disease. The PMMA fills the voids in the vertebra thereby reinforcing it. In some cases, additional cancellous bone, the bone inside the vertebra, may need to be removed thereby creating a void which can then be filled with PMMA. In other cases, it is the removal of a tumor that creates the void.

Kyphoplasty is a procedure similar to vertebroplasty whereby a balloon is inserted into the vertebral body and inflated to restore height to the bone if it has collapsed, most likely because of a fracture. After a space has been created the balloon is removed and the void is filled with PMMA. Other bio-compatible materials have also been clinically used and are under evaluation for filling these voids such as calcium sulfates and bone pastes.

Vertebroplasty and kyphoplasty are both typically performed using minimally invasive approaches and have been very successful for treating fractures and providing immediate pain relief. Unfortunately patients requiring such procedures often present with Adjacent Level Syndrome at a later date. In the case of vertebroplasty and kyphoplasty, adjacent vertebrae are typically subject to the same degenerative mechanisms which gave rise to the initial procedure. Since more than one vertebral body has now been affected, the need for spinal fixation, in addition to another vertebroplasty or kyphoplasty, might be necessary.

For this reason, it is undesirable to perform a subsequent procedure on the initially addressed vertebrae. Prior methods and devices for treating vertebral bodies or fixating the spine, only address either the delivery of the material into the bone or the fixation of the bone. Even though prior methods and devices are minimally invasive they require following each step of that procedure. By employing a device that is multi-functional, many steps can be eliminated while improving the likelihood for a successful outcome.

Thus, there is a need to provide improved surgical implants, instrumentation and minimally invasive methods for accessing a vertebral body, preparing the vertebral body by removing the damaged or diseased portions, delivering biomaterials and finally fixating a portion of the spine at the same time or as a subsequent action.

### SUMMARY OF THE INVENTION

The present invention is a unique minimally invasive surgical system and method for introducing instruments and/or biomaterial into the interior of a bone, particularly the interior of a vertebral body, using a unique cannulated screw that is sized and configured to penetrate the cortical bone and thereby provide access to the interior of the bone through the integral cannula of the screw. That same screw having a screw head that is then adaptable to employ a connector for securely holding a connecting device, such as a spinal rod or plate that can then be used to fix bones or bone fragments together. This unique system eliminates the need for a patient to undergo two separate procedures: first, that of penetrating the bone for the purpose of introducing biomaterials, such as bone cement, into the bone; and second, undergoing a separate procedure that introduces a separate bone screw into the bone so as to attach a connecting rod to the bone. The unique system and device of the present invention permits the procedures to be sequentially accomplished as a combined procedure with less trauma for the patient in a shorter surgical time period.

Thus, it is an object of the present invention to provide a minimally invasive method and device to address vertebral bodies of the spine and to access the interior of the vertebral bodies to correct injury and disease through the use of a unique cannulated bone screw.

It is further an object of the present invention to provide a screw having an integral cannula that has a diameter that is adequate in size to permit the introduction of at least one other instrument or device through the cannulation into the interior of the bone.

It is further an object of the present invention to provide a screw having an integral cannula wherein a portion of the length of the cannula lumen is provided with an opening through a portion of the screw shaft such that in that portion no screw threads are provided thus providing an opening from the lumen of the cannula to the exterior of the screw.

It is further an object of the present invention to provide a screw having an integral cannula that has a diameter that is adequate in size to permit the introduction of at least one other instrument or device through the cannulation, the cannulation having a portion that opens through the shaft of the screw to provide an opening from the lumen of the cannula to the exterior of the screw that is of adequate size and configuration to permit the at least one other instrument to pass out of the lumen of the cannula and to the exterior of the screw.

It is further an object of the present invention to provide a cannulated screw having an interior lumen of adequate size and configuration to permit through passage of at least one other instrument and having a screw head of low profile so as to not interfere with surround tissues in the body of the subject in which the screw is used.

It is further an object to provide the cannulated screw of the present invention having a screw head that is adapted to accept and hold a connection element such as a connection rod or plate.

It is further an object to provide the cannulated screw of the present invention wherein the head of the screw is capable of being connected to a spinal rod connector that in turn can be connected to a spinal rod or plate.

It is further an object of the present invention to provide a system for providing a minimally invasive method for accessing a vertebral body, preparing the vertebral body by removing the damaged or diseased portions, delivering biomaterials and finally fixating a portion of the spine using a unique cannulated bone screw.

Also provided is a method of using the novel cannulated screw of the present invention wherein the surgical method permits the use of the cannulated screw for penetrating and attaching to a bone for the purpose of introducing instruments or materials into the interior of the bone and subsequently attaching that same screw to a connecting device such as a spinal rod.

It is further an object of the present invention to provide a kit for use in accessing the interior of a bone for the purpose of introducing at least one instrument through the cannula of a unique cannulated bone screw and using that same cannulated bone screw as an element in the sequential method of fixing that same bone to a connecting device, such as a connecting rod or plate.

### BRIEF DESCRIPTION OF THE DRAWINGS

The foregoing and other features of the present invention will become apparent to one skilled in the art to which the present invention relates upon consideration of the following description of the invention with reference to the accompanying drawings, wherein:

FIG. 1 shows the cannulated screw access system of the present invention.

FIG. 2 shows an isometric view of a cannulated bone screw according to the present invention, the lumen of the cannula having a lateral opening along the threaded shaft of the bone screw;

FIG. 3 shows a front view of the cannulated bone screw of FIG. 1-2;

FIG. 4 shows a side view of the cannulated bone screw of FIGS. 1-3;

FIG. 5 shows a cross-sectional view of the cannulated bone screw of FIGS. 1-4;

FIG. 6 shows a cross-sectional view of the cannulated bone screw of FIGS. 1-5 having an instrument passed through and out of the lumen of the cannula;

FIG. 7 shows a top view of the cannulated bone screw of FIGS. 1-6 showing the upper access portal to the lumen of the cannulae and lateral opening of the cannula on the side of the threaded shaft of the screw;

FIG. 8 shows a front view of a cannulated bone screw according to the present invention, the lumen of the cannula having a distal opening along the longitudinal axis of the threaded shaft of the bone screw;

FIG. 9 shows a side view of the cannulated bone screw of FIG. 8;

FIG. 10 shows a cross-sectional view of the cannulated bone screw of FIGS. 8-9;

FIG. 11 shows a top view of the cannulated bone screw of FIGS. 8-10;

FIG. 12 shows a cross-sectional view of the cannulated bone screw of FIGS. 8-11 having an instrument passed through and out of the lumen of the cannula;

FIG. 13A shows the side view the cannulated bone screw of FIGS. 8-12 having an attached head for connection to a connecting rod; such as a spinal rod;

FIG. 13B shows a cross-sectional view of the cannulated bone screw of FIG. 13A;

FIG. 14 shows an isometric view of a vertebral body with a cannulated bone screw with a lateral opening threaded into the pedicle with an instrument passing down the cannulation and through the lateral opening into the bone;

FIG. 15 shows a front view of a vertebral body with a cannulated bone screw with a lateral opening threaded into the pedicle with an instrument passing down the cannulation and through the lateral opening into the bone.

### DETAILED DESCRIPTION OF THE INVENTION

Detailed embodiments of the present invention are disclosed herein; however, it is understood that the following description is provided as being exemplary of the invention, which may be embodied in various forms without departing from the scope of the claimed invention. Thus, the specific structural and functional details provided in the description are non-limiting, but serve merely as a basis for the invention defined by the claims provided herewith.

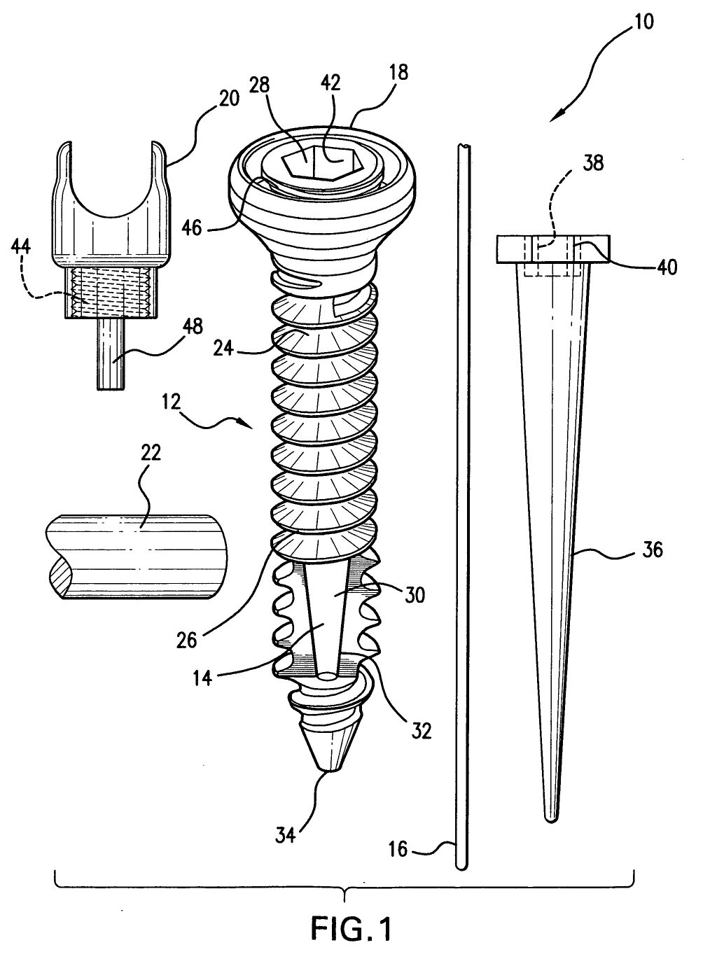

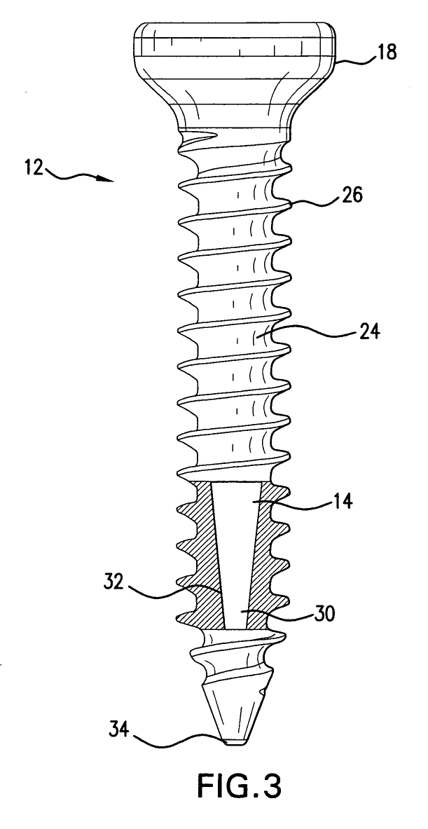

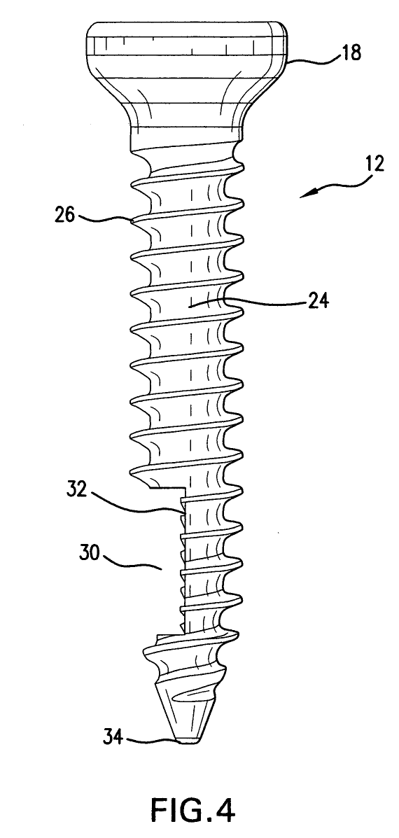

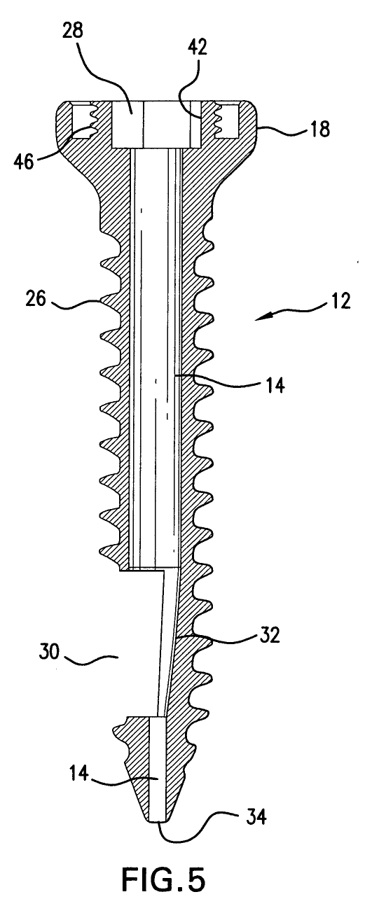

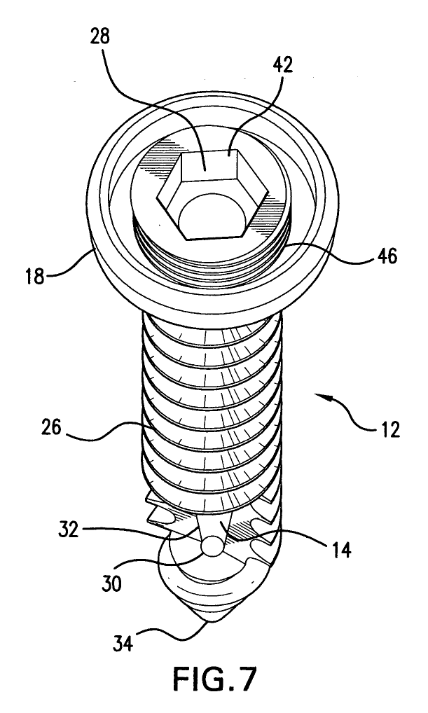

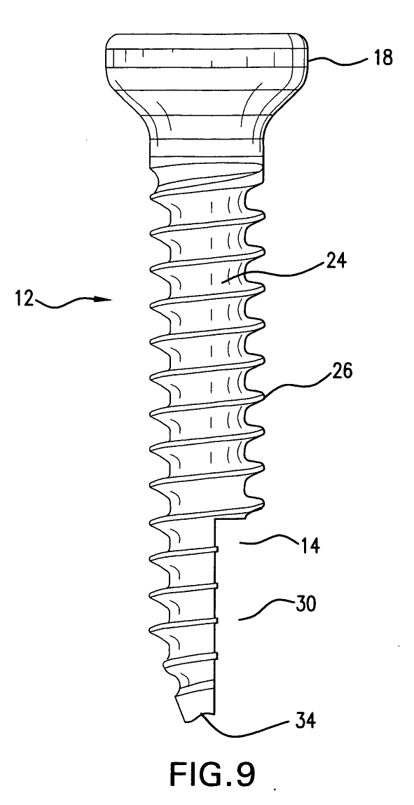

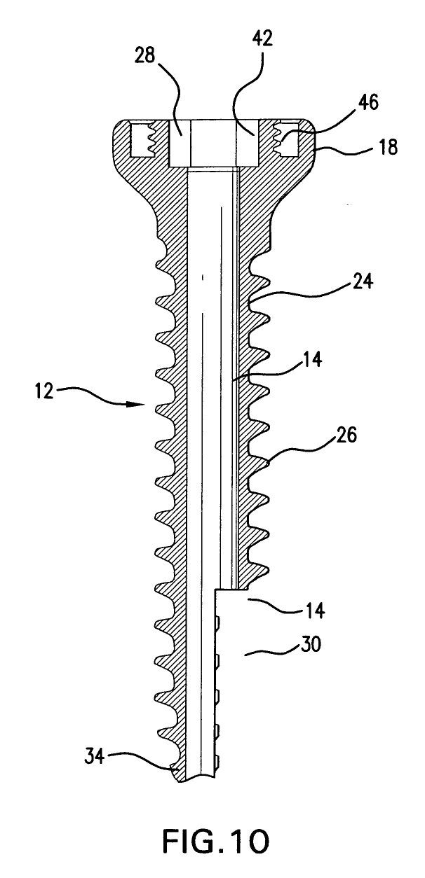

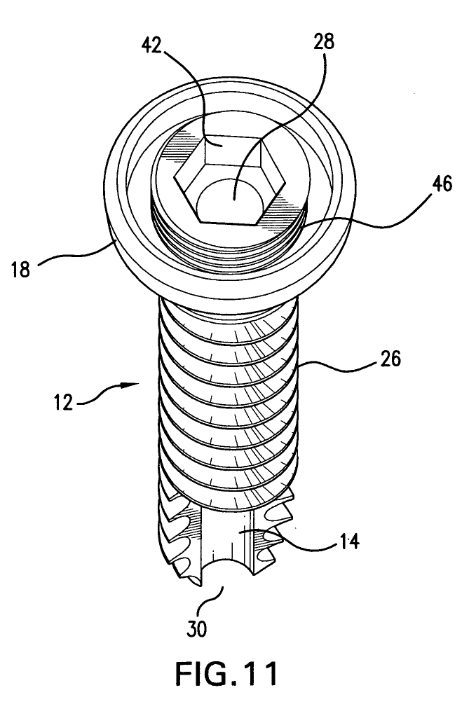

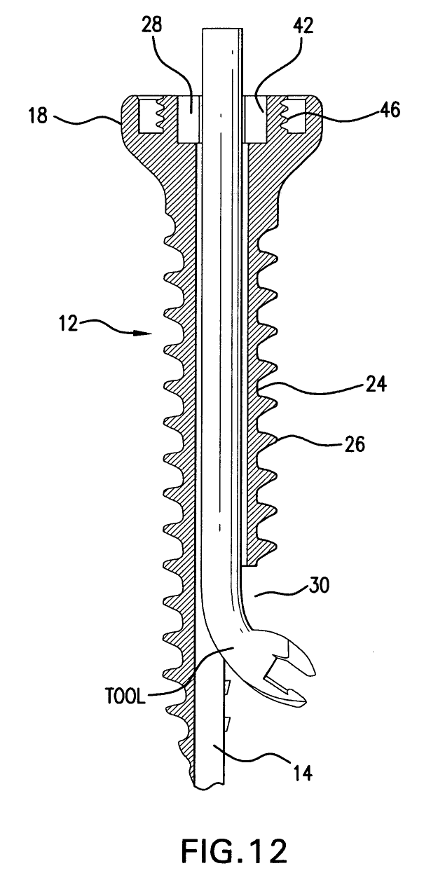

As shown in FIGS. 1-15, a novel cannulated screw access system, generally shown at 10, includes a unique cannulated screw 12 that is sized and configured to be capable of penetrating cortical bone and providing an integral cannula lumen 14 for accessing the interior of the bone, the cannulae lumen 14 being sized to facilitate the passage of instruments or biomaterials into the interior of the bone. A guide element 16, such as a guide wire or cannula, may be positioned at the surgical site prior to inserting the cannulated screw 12 along the pathway of the guide element 16 to facilitate proper trajectory of the cannulated screw insertion process. The screw head 18 of the bone screw 12 is configured to receive a head adapter 20 configured to releasably secure a connecting element 22, such as a spinal rod or plate, thereby enabling the cannulated bone screw 12 to first be used to access the interior of the bone to introduce instruments or biomaterials and then to be securely in the bone and used as a bone screw for fixating bones or fragments of bones.

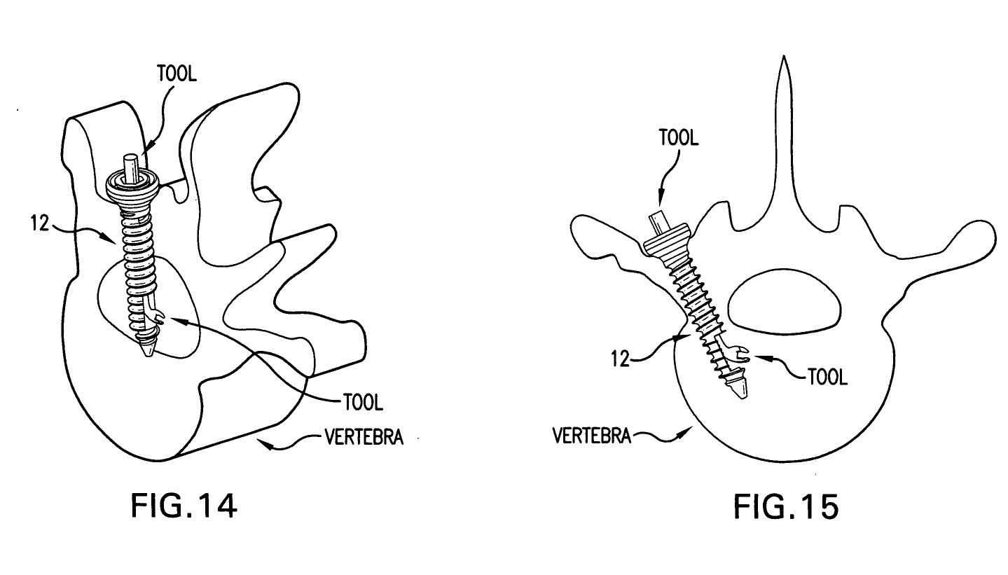

The cannulated screw 12 includes a screw shaft 24 having threads 26, which are sufficient to penetrate and be securely fixed into bone, specifically the pedicle of a vertebral body, as demonstrated in FIGS. 14-15. The length of the screw shaft 24 can vary such that it achieves unicortical or bicortical fixation.

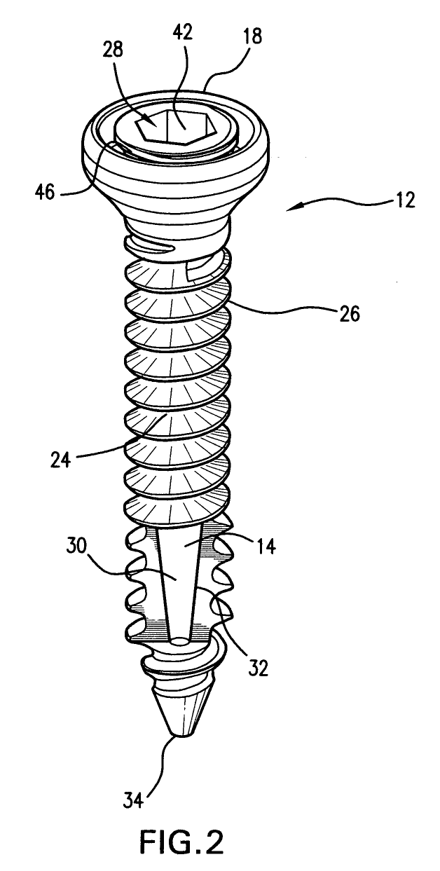

The screw shaft 24 defines the lower portion of the cannula lumen 14, the upper portion of which extends through the screw head 18 to a cannula lumen ingress portal 28 defined by the screw head 18. As best shown in FIGS. 2-13B a cannula lumen egress portal 30 can be defined in the wall of the cannula lumen 14 so as to allow the movement of instruments or materials from a first location outside the cannulated screw 12 and through the cannula lumen 14 to a second location outside the cannulated screw 12. The egress portal 30 can be defined in the side wall 32, as shown in FIGS. 2-7, of the cannula lumen or alternatively at the lower or distal end 34 of the cannulated screw 12. The cannula lumen 14 can be configured to have a decreasing tapered diameter or a constant diameter for the entire length of the cannula lumen 14. A lateral opening through the side wall of the screw shaft 24 for the egress portal can be best suited to a decreasing tapered diameter cannula lumen in that the taper aids in directing an instrument or material in the lumen to and out of the egress portal 30. The embodiment of the cannulated screw having an egress portal 30 at the distal end 34 of the cannulated screw 12 is best suited for a constant diameter cannula lumen 14.

As best shown in FIGS. 2-7, when the egress portal 30 is in the side of the cannula lumen, it forms an opening in the side of the screw shaft 24 and interrupts the screw threads 26 at that position. The remainder of the screw shaft 24 is threaded and those threads remain in normal alignment from one side of the egress portal 30 to the other in order to facilitate the threading of the screw into the bone with minimal interference. The distal end 36 of the cannula lumen 14 of a cannulated screw 12 having a egress portal 30 defined in the side of the screw shaft 24 can be angled or curved so as to facilitate ease of movement of any instrument or material from the cannula lumen 14 to a location outside the cannulated screw 12.

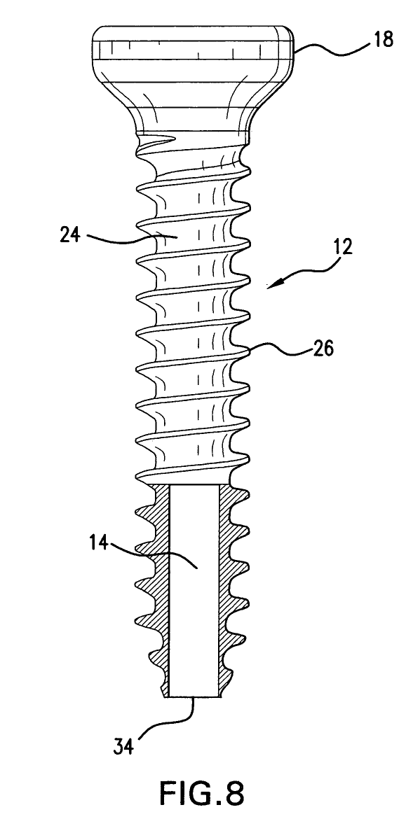

As best shown in FIGS. 8-13B, the when the egress portal 30 is defined at the lower most part or distal end 34 of the cannulated screw, the cannulated screw does not have a penetrating point as is found in the side egress embodiment shown in FIGS. 2-7. The distal end 34 of the cannulated screw defines the lowest part of the egress portal 30 and as shown in FIGS. 8-13B, the egress portal 30 extends from that distal end 34 of the cannulated screw to a position part way up the screw shaft 24 thus opening both the cannula lumen 14 at a position on the bottom of the screw 24 shaft and contiguous with a lower portion of the side of the screw shaft 24. It is also within the concept of the present invention to provide this embodiment of the invention having only a distal end of the cannulated screw 34 egress portal 30 without also providing therewith the contiguous lateral opening in the lower part of the screw shaft 24.

As shown in FIG. 1, a core element 36 can be provided and optionally used to fill the void of the cannula lumen 14 during the insertion or if desired during the removal of the screw from the bone. The core element 36 is sized configured to maintain the open space of the cannula lumen 14 as the cannulated screw 12 is inserted into the bone and by doing so to help prevent the coring of the bone or the unwanted filling of the cannula lumen with tissue or bone material during the process of inserting the cannulated screw 12. The core element 36 can be provided with proximally located internal and external gripping surfaces 38, 40, to facilitate releasable engagement of the core element 36 with the screw head 18 and with a tightening or loosening tool used by the surgeon. The core element 36 internal and external gripping surfaces 38, 40 by releasable engagement with the surgeons tool and the head of the screw 18, enables the surgeon to have the core element in place within the lumen 14 of the cannulated screw during the screw insertion or removal process while the surgeon applies tightening or loosening torque to the screw head 18 as transmitted through the connection of the core element 36. The guide element 16, as shown in FIG. 1, can be configured to serve as a core element 36 for a cannulated screw having the egress portal 30 located at the distal end of the cannulated screw 34.

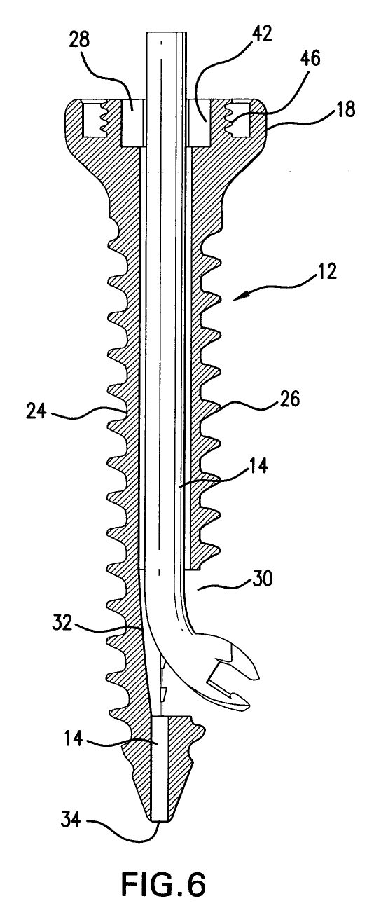

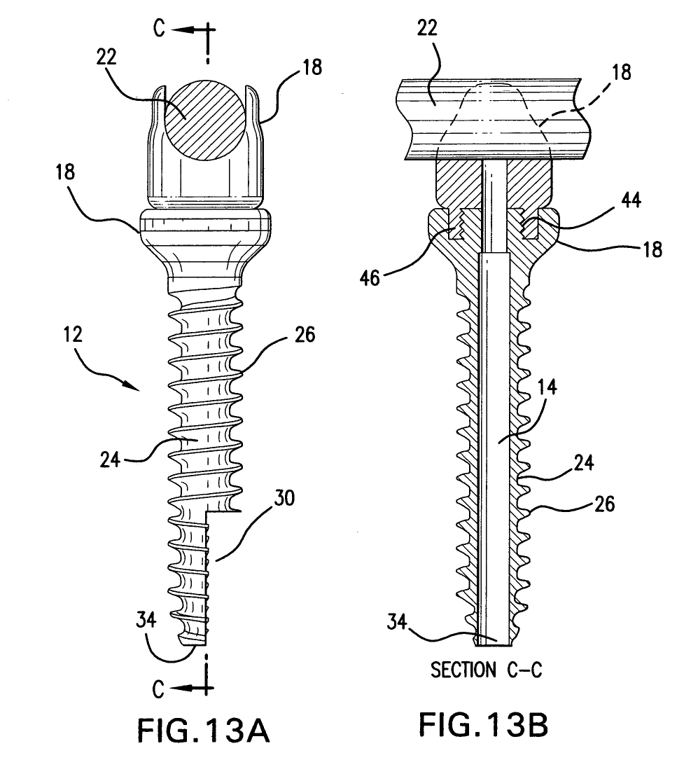

The screw head 18 of the cannulated screw 12 is provided with gripping surfaces 42 that can be such that a standard tightening or loosening tool such as one having a hex or square drive can be attached to drive the cannulated screw 12 into or out of the bone. Any other configuration for the screw head gripping surfaces 42 can be employed provided they have a geometry complementary to that of the tightening or loosening tool used by the surgeon. The screw head 18, when fully inserted into the bone has a very low profile so as to not excessively protrude or interfere with surrounding anatomy and tissue. As best shown in FIGS. 1, 13A, and 13B, the head adaptor 20 can be attached to the screw head 18 for purposes of providing a means to secure a connecting element 22, such as a spinal rod or plate to the cannulated screw 12. It is within the concept of the present invention for the head adaptor 20 to be releasably attached, fixedly attached, or integrally formed to the screw head 18. If the head adaptor 20 is attached to the screw head 18, it can be accomplished by providing the head adaptor 20 and the screw head 18 with complementary configured adaptor attachments 44, 46, which preferably are complementary threaded surfaces as shown in FIGS. 6-7, 10-12, and 13B. The head adaptor 20 can be provided in any configuration that facilitates the attachment of orthopedic devices to the cannulated screw 12; such as, for example adaptors suited for spinal rods, spinal plates, vertebral cross-connector, or the like. It is within the concept of the present invention for the head adaptor 20 to be configured as a universal attachment connector, such as a threaded shaft, snap fit shaft, bayonet shaft, or any other suitable connector that would be suitable to attach any of a wide variety of devices to the screw head of the present invention. For example, the head adaptor 20 can be provided with a threaded stem such as shown and described in U.S. Pat. No. 5,735,851, the complete disclosure of which is herein fully incorporated by reference. An adaptor guide 48 can be provided to facilitate correct adaptor placement onto the screw head 18. The adaptor guide 48 can be sized and configured to fit within the upper part of the cannula lumen 14 and thereby guide the complementary adaptor attachments 44, 46 into alignment.

A surgeon using the present invention can position a guide element 16, such as a guide wire or cannula, at the surgical site prior to inserting the cannulated screw 12 along the pathway of the guide element 16 to facilitate proper trajectory of the cannulated screw insertion process. The use of a guide element 16 may not be required in all cases. Once properly positioned at the surgery site, the surgeon can apply force to the screw head 18 of the cannulated screw so as to enable penetration of the threaded screw shaft 24 through the cortical bone and into the interior of the bone. If the surgeon has employed a core element 36 to inhibit obstruction of the lumen 14 of the cannulated screw 12, it can be removed to permit the surgeon to insert at least one instrument or to insert material into the interior of the bone as needed. Upon completion of the surgeon's treatment of the bone, a head adaptor 20 can be attached to the screw head 18, if desired. Using the head adaptor 20, the surgeon can then attach any of a wide variety of devices to the securely anchored screw 12. As a non-limiting example, a spinal rod or plate can be attached to the head adaptor 20.

The materials used to construct the present invention are those which have sufficient strength, resiliency, and biocompatability as is well known in the art for such devices. Methods of manufacture of such surgical implant devices is also well known in the art.

It is within the concept of the present invention to provide the cannulated screw access system 10 as part of a kit for use in a surgical process, the kit comprising at least one of the screws 10 and at least some of the associated tools for using the screws to connect a surgical rod to adjacent bones or bone fragments. In addition, the kit can contain surgical rods, such as, for example, spinal rods. Additional devices such as cross-connectors or links can also be included in the kit.

Each of the embodiments described above are provided for illustrative purposes only and it is within the concept of the present invention to include modifications and varying configurations without departing from the scope of the invention that is limited only by the claims included herewith.

What is claimed is:

1. A novel cannulated screw access system, the system comprising:
a cannulated screw having a proximal first end and a distal second end, said screw having a screw head at said first end and a shaft being at least partially threaded extending from said screw head to said second end, said screw having a cannula lumen extending from a cannula lumen first portal at said first end to a cannula lumen second portal defined in a portion of said screw shaft, said cannula lumen being sized to permit movement of at least one instrument or movement of material through said cannula lumen;
a head adaptor configured to be fixed to said screw head, said head adaptor being capable of attachment to another orthopedic device;

2. The system of claim 1, further comprising a guide element capable of serving as a guide for said cannulated screw, said guide element being sized and configured to permit passage of said guide wire through said lumen.

3. The system of claim 1, wherein said second portal is defined in the side of said screw shaft such that said second portal opens said lumen to the outside of said screw shaft at a position proximal to said second end.

4. The system of claim 3, wherein said cannula lumen is configured as a decreasing taper from said first portal to said second portal.

5. The system of claim 1, wherein said second portal is defined at the second end of said screw.

6. The system of claim 1, wherein said cannula lumen is of constant diameter.

7. The system of claim 5, wherein said second portal is elongated to extend from said second end of said screw to a position on the side of said screw shaft.

8. The system of claim 1, wherein said screw head comprises gripping surfaces complementary to the configuration of a tool for applying torque to said screw head.

9. The system of claim 1, wherein said screw head defines a head adaptor receiver configured to fixedly receive said head adaptor.

10. The system of claim 9, wherein said adaptor for said screw head and said head adaptor receiver have attachment surfaces with complementary geometry.

11. The system of claim 10, wherein said attachment surfaces having complementary geometry are selected from the group consisting of threaded, bayonet, snap fitting, friction fit, and luer lock.

12. The system of claim 10, wherein said head adaptor comprises an adaptor guide element positioned on the undersurface of said head adaptor and configured to facilitate alignment of said head adaptor to said screw head.

13. The system of claim 12, wherein said adaptor guide is an elongated structure descending vertically from the underside of said head adaptor and sized and configured to slidably pass into the upper portion of said first portal of said cannula lumen.

14. The system of claim 3, wherein said cannula lumen terminates with an angle or curve toward said second portal so as to facilitate passage of instruments or material out of and into said cannula lumen through said second portal.

15. The system of claim 1, further comprising a core element, said core element being sized and configured to occupy the open space of said cannula lumen and to be selectively removable from said lumen.

16. The system of claim 1, wherein said head adaptor is integrally formed with said screw head.

17. The system of claim 1, wherein said head adaptor is permanently fixed to said head adaptor.

18. The system of claim 1, wherein said head adaptor is releasably fixed to said head adaptor.

19. A cannulated screw comprising;
a screw having a proximal first end and a distal second end, said screw having a screw head at said first end and a shaft being at least partially threaded extending distally from said screw head toward said second end,
a cannula lumen defined within and along the longitudinal axis of said screw, said lumen extending from a cannula lumen first portal at said first end to a cannula lumen second portal defined in a portion of said screw shaft, said cannula lumen being sized to permit movement of at least one instrument or movement of material through said cannula lumen.

20. The cannulated screw of claim 19, wherein said second portal is defined in the side of said screw shaft such that said second portal opens said lumen to the outside of said screw shaft at a position proximal to said second end.

21. The cannulated screw of claim 20, wherein said cannula lumen is configured as a decreasing taper from said first portal to said second portal.

22. The cannulated screw of claim 19, wherein said second portal is defined at the second end of said screw.

23. The cannulated screw claim 19, wherein said cannula lumen is of constant diameter.

24. The cannulated screw of claim 22, wherein said second portal is elongated to extend from said second end of said screw to a position on the side of said screw shaft.

25. The cannulated screw of claim 19, wherein said screw head comprises gripping surfaces complementary to the configuration of a tool for applying torque to said screw head.

26. The cannulated screw of claim 19, wherein said screw head defines a head adaptor receiver configured to fixedly receive a head adaptor.

27. The cannulated screw of claim 26, wherein said head adaptor receiver comprises attachment surfaces that are of complementary geometry to attachment surfaces of a head adaptor.

28. The cannulated screw of claim 27, wherein said attachment surfaces having complementary geometry are selected from the group consisting of threaded, bayonet, snap fitting, friction fit, and luer lock.

29. The cannulated screw of claim 20, wherein said cannula lumen terminates with an angle or curve toward second portal so as to facilitate passage of instruments or material out of and into said cannula lumen through said second portal.

30. The cannulated screw of claim 19, further comprising a core element, said core element being sized and configured to occupy the open space of said cannula lumen and to be selectively removable from said lumen.

31. A kit for accessing the interior of a bone and for providing a bone screw anchor in said bone, said kit comprising:
the cannulated screw access system of claim 1;
at least one tool for use with said cannulated screw access system.

32. The kit according to claim 31, further comprising at least one additional orthopedic device.

33. The kit according to claim 32, wherein said at least one orthopedic device is selected from the group consisting of a connecting rod, a bone plate, and a trans-vertebral bone connecting device.

34. A method for treating an injury or pathology of a bone requiring fixating to another bone or bone fragment, the method comprising:
obtaining the cannulated bone access system of claim 1;
connecting said cannulated bone screw to a bone of a subject;
accessing the interior of said bone with at least one instrument or a material to be introduced into said interior of said bone;
treating said bone using said at least one instrument or said material to be introduced into said bone;
attaching said head adaptor to said cannulated screw;
attaching at least one orthopedic device to said head adaptor.

35. The method of claim 34, wherein said bone being treated is a vertebra.

36. The method of claim 35, wherein said at least one orthopedic device is selected from the group consisting of a spinal rod, a bone plate, and a trans-vertebral cross-connector.

37. The method of claim 35, wherein said material to be introduced is biocompatible.

38. The method of claim 36, wherein said material to be introduced in bone cement.
